(19) United States
(12) Patent Application Publication
(10) Pub. No.: US 2008/0260642 A1
(43) Pub. Date: Oct. 23, 2008

(54) Bacteriophage-Based Contrast Agents, the Use Thereof and a Method for the Production Thereof

(76) Inventor: Arne Hengerer, Erlangen (DE)

Correspondence Address: HARNESS, DICKEY & PIERCE, P.L.C., P.O.BOX 8910, RESTON, VA, 20195, US

(21) Appl. No.: 11/885,040

(22) PCT Filed: Feb. 22, 2006
(86) PCT No.: PCT/EP06/60186

§ 371 Date: Aug. 24, 2007

(30) Foreign Application Priority Data
Feb. 25, 2005 (DE) 10 2005 008 765.5

(51) Int. Cl. A61K 49/06; C12N 15/00; A61K 35/66; C12P 21/06; A61K 49/00; A61K 49/04

(52) U.S. Cl. 424/9.1; 435/320.1; 424/93.6; 424/9.4; 424/9.3; 435/69.7

(57) ABSTRACT

Bacteriophage-based contrast agents and a method for the production thereof are disclosed. The bacteriophages, in at least one embodiment, contain a first fusion protein comprising a phage-surface protein and a ligand specific for an identifiable target structure and a second fusion protein comprising a phage-surface protein and a peptide binding a signal generating molecule.

The present invention relates to novel bacteriophage-based contrast agents, to the use thereof and to a method for the production thereof.

Contrast agents for molecular imaging usually consist of a signaling molecule, a ligand which is capable of binding to a target structure to be detected, and a linker for coupling the signaling molecule and the ligand.

Signaling molecules already in use in the prior art are radioisotope-labeled molecules, molecules labeled with ferro-, para- or supramagnetic elements, fluorophores or the like. Known examples are biomolecules in which a stable isotope has been replaced with a positron emitter such as, for example, ¹¹C, ¹³N or ¹⁵O. This enables the metabolic behavior of the labeled biomolecules to be monitored. Magnetic resonance tomography (MRT) makes use of para- or ferromagnetic substances such as, for example, chelated Gd or iron oxide nanoparticles as signaling molecules.

The ligand is a structure capable of binding specifically to a target structure to be detected. The target structure to be detected may be any suitable marker, for example a disease marker, i.e. a highly specific marker for a pathologically modified tissue such as, for example, tumors, inflammatory foci etc.

The ligands are therefore, for example, specific peptides such as antibodies or fragments thereof, for example. The ligand is currently obtained in complicated processes such as, for example, high throughput processes, isolated from chemical or biological libraries, obtained as antibody after immunization or prepared by “rational design”.

Bacteriophages have been used in the prior art for surface display and for selecting peptides and polypeptides. An example of this is the phage display technique (see, for example, Hoogenboom H. R., “Overview of Antibody Phage-Display Technology and its applications”, Methods of Molecular Biology, 2002, Vol. 178, pp. 1-37, and Ladner, R. C., “Polypeptides from phage display. A superior source of in vivo imaging analysis”, The Quarterly Journal of Nuclear Medicine, June 1999, Vol. 43(2), pp. 119-124).

Phages most frequently used for phage display technique in the prior art are the bacteriophages M13, f1 and Fd which belong to the filamentous phages, Ff.

Conventional phage display involves firstly generating a library of phage DNA, wherein a number of DNA sequences to be selected are cloned into phage genomes or else modified phage genomes similar to plasmids, also called phagemids. This involves cloning the DNA sequence to be selected into the phage genome in such a way that expression results in a fusion protein of one of the phage surface proteins, usually gpIII, gpVIII or gpVI, and the amino acid sequence encoded by the DNA sequence cloned therein. The phage genome library is then introduced into a suitable host cell, where recombinant phage particles are generated which display fusion proteins on their surface.

Selection techniques (e.g. panning, affinity maturation) are then used for identifying those phage genomes from the library that include DNA sequences whose encoded peptides or polypeptides have particular binding properties.

Panning involves contacting the recombinant phage particles obtained from the phage genome library with a target structure and selecting those phage particles having the desired binding properties. Positive selection comprises isolating those phage particles which bind to the target structure. Negative selection on the other hand comprises discarding those phages which bind to a structure other than the target structure. Multiple selection cycles are carried out frequently.

The prior art uses affinity maturation primarily for selecting antibodies or antibody fragments. This involves carrying out, after the first selection, modifications in the nucleic acid regions coding for the variable region of antibodies (V). These modifications result in further diversification of the V region, and a secondary library is generated based on the modified nucleic acid sequences obtained in this way. The latter library is then once more subjected to the selection processes mentioned.

Conventional contrast agents are often not specific or not specific enough. Another disadvantage of conventional contrast agents is inter alia the fact that coupling of the ligand to the signaling molecule often alters the binding properties of the ligand. In many cases this results in the ligand bound to the signaling molecule having other, and usually poorer, binding properties than the uncoupled ligand. In many cases the ligand has a considerably reduced affinity for the target structures to be detected. It is even possible for undesired cross reactivities of the ligand to arise after coupling of said ligand to the signaling molecule.

The object addressed by the present invention is to provide a novel contrast agent which avoids the disadvantages mentioned. WO 00/32625 describes hepatitis B virus (HBV) particles containing fusion proteins of a peptide binding to an HBV capsid and an immunogen. These particles are intended to increase the immune reaction to the immunogen present therein. Optionally the peptides binding to the HBV capsid may include markers.

Said object is achieved according to the invention by a contrast agent based on a recombinant phage particle, comprising a first fusion protein which comprises a phage surface protein and a ligand specific for a target structure to be detected, and a second fusion protein which comprises a phage surface protein and a peptide or polypeptide, wherein said peptide or polypeptide is a signaling molecule and/or is capable of binding to a signaling molecule.

Suitable phage particles are in principle any phages useful for phage display, i.e. phages that can display fusion proteins of phage surface proteins and foreign peptides or polypeptides on their surface. Preference is given in particular to Inoviridae and specifically to the filamentous bacteriophages M13, f1 and fd.

The ligand is a peptide or polypeptide which has specificity for binding to a particular target structure.

A target structure means in accordance with the present invention any target structure suitable for detection. This may be, for example, disease markers or else molecules present on particular tissues but not on other tissues. They may also be any kind of surface markers. Particular preference is given here to specific disease markers such as, for example, cell surface markers of tumor cells (e.g. proliferation markers, CEA), markers for vulnerable plaques (e.g. MMP, matrix metalloproteases), endothelial neoangiogenesis markers (e.g. VEGF), rheumatoid arthritis markers (e.g. matrix metalloproteases).

The ligand is preferably a linear peptide or polypeptide, a cyclic peptide or polypeptide, or an antibody or antibody fragment, for example an Fab fragment. It is also possible to use merely individual parts of the variable (V) regions of the heavy and light chains of antibodies as ligand (VL, VH). Suitable ligands are both natural and synthetic peptides and polypeptides.

The peptide or polypeptide which is a signaling molecule or is capable of binding to such a molecule may be any suitable peptide or polypeptide.

If the peptide or polypeptide itself is the signaling molecule, it may be detected by a separate detection reagent, for example owing to its specific structure, after binding of the ligand to the target structure.

Preference is given, however, to the peptide or polypeptide being capable of binding to a signaling molecule.

Examples of suitable signaling molecules are radioisotope-labeled molecules, molecules labeled with para-, ferro- or supramagnetic elements, for example a chelated lanthanide such as Gd, and fluorophores. Examples of fluorophores are GFP (green fluorescence protein), DsRed (red fluorescence protein), CY5.5, CY7 or indocyanine green.

Preference is given to the recombinant phage particles of the invention presenting in each case different phage surface proteins as fusion proteins with the peptide or polypeptide specific for the signaling molecule and the ligand.

If a filamentous phage such as, for example, M13, fd or f1 is used, the surface proteins gpIII, gpVI and gpVIII are particularly suitable for expressing fusion proteins.

Particular preference is given to the ligand being expressed as fusion protein with gpIII, for example via the fUSE expression vector. Only about three to five copies of gpIII are present on the phage surface. Moreover, gpIII enables the ligand to be displayed locally at only one end of the phage particle.

It is however preferred for the peptide or polypeptide capable of binding to a signaling molecule to be displayed on the phage surface in as large a number as possible. Therefore preference is given to expressing said peptide or polypeptide as fusion protein with the phage surface protein gpVIII, for example via the f88 expression vector. This results in said peptide or polypeptide being expressed virtually across the entire surface of the phage particle, thus allowing a high load of signaling molecules.

One advantage of a contrast agent of the invention based on recombinant phage particles consists of the fact that the phage particle is used as a linker between the signaling molecule and the ligand. This makes it possible firstly to avoid unspecific binding of signaling molecules to the ligand and thereby an undesired deterioration of the binding properties of said ligand. Furthermore, the phage display technique also provides the possibility of preparing a biomolecular contrast agent allowing a high load of signaling molecules.

The contrast agents of the invention may be applied in principle to any conventional imaging analyses such as, for example, X-ray computed tomography (CT), magnetic resonance tomography (MRT), positron emission tomography (PET), single photon emission computed tomography (SPECT) or optical imaging such as near infrared fluorescence (NIRF).

The present invention furthermore provides a method for producing a contrast agent based on recombinant phage particles, wherein said phage particles have a signaling molecule or are capable of binding to a signaling molecule and whose surface has a ligand specific for a target structure to be detected, wherein said method comprises the steps of:

(a) providing a library of recombinant phage genomes comprising
a first nucleic acid sequence coding for a fusion protein of a first phage surface protein and a ligand to be selected, and a second nucleic acid sequence coding for a fusion protein of a second phage protein and a peptide, wherein said peptide is a signaling molecule or is capable of binding to a signaling molecule,
(b) introducing the recombinant phage genomes of (a) into a suitable host cell and expressing said phage surface genomes to generate recombinant phage particles,
(c) where appropriate, selecting phage particles which have peptides with specificity for a signaling molecule,
(d) selecting phage particles which have ligands with specificity for said target structure to be detected, and
(e) isolating the selected phage particles.

Step (a) comprises conventional cloning techniques for phage display which are known to the skilled worker. Firstly, a ligand library is selected. This is preferably a library of nucleic acid sequences coding for antibody fragments.

Furthermore, a second nucleic acid sequence or a corresponding library is selected which codes for the peptide intended to bind specifically to a signaling molecule.

Both nucleic acid sequences or nucleic acid sequence libraries are then cloned into appropriate phage genomes. It is possible here to use conventional kits for phage display, such as, for example, the PRAS system from Amersham (now GE Healthcare), the PhD Peptide Display Kit from New England Biolabs, the Phage Display Kit by Creg Winter or else conventional phagemids.

Preference is given to “miniphagemids” which are deleted mutants containing only 20 to 50% of the wild type phage genome and thus are distinctly shorter than wild type phages (wild type phages are about 900 nm in length and 9 nm in diameter).

Step (b) involves propagating the phage genomes in suitable host cells, with known bacteria strains such as, for example, E. coli, in particular the E. coli strains K802, K91 or MC1061, being particularly suitable here.

In step (c) those phage particles of the library which have the desired peptides or polypeptides and ligands with particular binding properties are identified by the selection techniques mentioned at the outset (e.g. panning, affinity maturation).

If a specific peptide capable of binding to a signaling molecule is already known, a selection for this peptide is not required.

It is also possible to select for such a peptide. Such a selection may comprise the following:

In the case of a fusion protein with the peptide or polypeptide capable of binding to a signaling molecule, preference is given to using a

phage surface protein of which a large number of copies are expressed, for example the surface protein gpVIII (major coat protein, 2700 to 3000 copies per phage particle). A fusion protein of the surface protein and an amino-terminally fused peptide is permutated at the foreign peptide in order to generate a phage library. This involves employing common genetic engineering methods for preparing phage libraries. Panning is used to select for specificity to a signaling molecule (e.g. radioisotopes, lanthanides, fluorophores, derivatized forms of the signaling molecules above, which are bound to chelates or coupling groups, etc.). The phages obtained in this way are propagated in suitable cultures (bacteria, cell culture) and, where appropriate, accumulated in multiple panning cycles to the same signaling molecule. If required, the binding properties are optimized by means of the usual affinity maturation techniques (described in the literature). The target structure-binding phages are amplified and incubated in a solution of the signaling molecule.

Subsequently, phage particles having ligands with specificity for the target structure to be detected may be selected as follows:

The enriched phage library is panned to a healthy tissue sample, for example a homogenate, from the target organ, which does not have the target structure to be detected. Those phages which bind to a structure other than the target structure (which thus exhibit cross reactivity) are discarded this time (negative selection).

The phage library which has already been selected twice is then permutated a second time, but this time at the gene which codes for the fusion protein between surface protein gpIII and amino-terminal foreign protein (e.g. linear peptide, cyclic peptide, antibody fragment). In the subsequent panning cycles those phage particles are isolated which bind to the target structure (positive selection). If

required, the binding properties are optimized by means of the common affinity maturation techniques described in the literature.

Preference is given to carrying out at least one positive and one negative selection. Preferably multiple selection cycles are carried out.

Since the peptide for the signaling molecule is already present during selection for the ligand, the binding properties of said ligand cannot be changed later, which is often the case with contrast agents of the prior art when the ligand is subsequently coupled to the signaling molecule.

Although the order of the selection steps is not fixed, preference is given to selecting first for the peptide binding to a signaling molecule and only then for the ligand.

The thus selected phage particles are then isolated. The phage particles thus isolated may then be loaded with a desired signaling molecule.

After appropriate purification, in particular after removing bacterial toxins, and loading with signaling molecules, the phages may be used directly as contrast agents.

An exemplary embodiment of a recombinant phage particle of the invention is depicted in the drawing in which

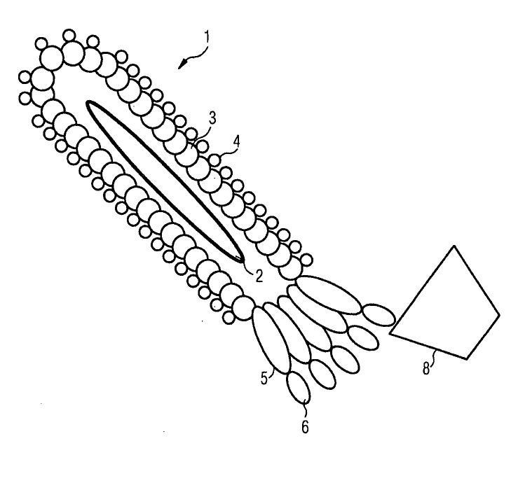

FIG. 1 depicts a diagrammatic representation of a recombinant phage particle.

The recombinant phage particle 1 has an envelope of surface proteins. Said envelope encloses the phage genome 2. The envelope comprises firstly a fusion protein of a surface protein 3, here gpVIII, and a peptide or polypeptide which is a signaling molecule 4 or binds to such a molecule. The envelope further comprises a fusion protein of a surface protein 5, here gpIII, and a ligand 6 specific for a target structure 8 to be detected. In the example shown, only 5 copies of the surface protein gpIII are present, and, as a result, the ligand 6 is present also only five times.

Particular advantages arise for the application of the above-described contrast agent in MRT imaging, since the latter is limited with respect to the lower detection limit. The above-described phage contrast agents have a high “pay load” (high ratio of signaling molecules to binding molecules). Theoretically, one phage may bind up to 3000 signaling molecules. This generates a very strong signal in the (pathological) target tissue, which in some cases makes only possible MR detection of the molecular markers of which only a limited number of copies is present.

The invention furthermore provides a diagnostic and/or therapeutic composition whose active substance is a contrast agent according to the invention. The composition furthermore comprises suitable additives and/or excipients.

It is possible to employ the contrast agent of the invention not only diagnostically but also therapeutically. To this end, either a therapeutic substance may be coupled to the ligand or the ligand itself is therapeutically active. Possible specific applications are in particular in tumor diagnostics and tumor therapy.

1. A contrast agent based on recombinant phage particles, comprising:
a first fusion protein including a phage surface protein and a ligand specific for a target structure to be detected; and
a second fusion protein including a phage surface protein and at least one of a peptide and polypeptide, wherein said at least one of a peptide and polypeptide is at least one of a signaling molecule and capable of binding to a signaling molecule.

2. The contrast agent as claimed in claim 1, wherein the first phage surface protein is gpIII.

3. The contrast agent as claimed in claim 1, wherein the second phage surface protein is gpVIII.

4. The contrast agent as claimed in claim 1, wherein the phage is selected from the group consisting of Inoviridae, M13, f1 and fd.

5. The contrast agent as claimed in claim 1, wherein the signaling molecule is selected from the group consisting of radioisotope-labeled molecules, molecules labeled with para-, ferro- or supramagnetic elements, and fluorophores.

6. The contrast agent as claimed in claim 1, wherein the ligand is selected from the group consisting of linear peptides or polypeptides, cyclic peptides or polypeptides, antibodies and antibody fragments.

7. At least one of a diagnostic and therapeutic composition comprising a contrast agent as claimed in claim 1 and at least one of suitable additives and excipients.

8. A method, comprising:
using the contrast agent as claimed in claim 1 in an imaging analysis in a patient.

9. The method as claimed in claim 8, wherein the imaging analysis is based on at least one of X-ray computed tomography (CT), magnetic resonance tomography (MRT), positron emission tomography (PET), single photon emission computed tomography (SPECT) and optical imaging such as near infrared fluorescence (NIRF).

10. A method of producing a contrast agent based on recombinant phage particles at least one of including a signaling molecule and capable of binding to a signaling molecule and whose surface has a ligand specific for a target structures to be detected, the method comprising:
(a) providing a library of recombinant phage genomes comprising a first nucleic acid sequence coding for a fusion protein of a first phage surface protein and a ligand to be selected, and
a second nucleic acid sequence coding for a fusion protein of a second phage surface protein and a peptide, the peptide being at least one of a signaling molecule and capable of binding to a signaling molecule;
(b) introducing the recombinant phage genomes of into a suitable host cell and expressing the phage genomes to generate recombinant phage particles;
(c) selecting, where appropriate, phage particles which have peptides with specificity for a signaling molecule;
(d) selecting phage particles which have ligands with specificity for the target structure to be detected; and
(e) isolating the selected phage particles.

11. The method as claimed in claim 10, wherein selecting in at least one of step (c) and step (d) takes place by way of at least one of panning and affinity maturation.

12. The method as claimed in claim 10, wherein a positive and a negative selection are carried out in at least one of step (c) and step (d).

13. The method as claimed in claim 10, wherein the signaling molecule is selected from the group consisting of radioisotope-labeled molecules, molecules labeled with para-, ferro- or supramagnetic elements, and fluorophores.

14. The method as claimed in claim 10, wherein the isolated phage particles are contacted with a signaling molecule.

15. The method as claimed in claim 10, wherein the first phage surface protein is gpIII.

16. The method as claimed in claim 10, wherein the second phage surface protein is gpVIII.

17. The method as claimed in claim 10, wherein the phage is selected from the group consisting of Inoviridae, M13, f1 and fd.

18. The contrast agent as claimed in claim 2, wherein the second phage surface protein is gpVIII.

19. A method, comprising:
using the composition as claimed in claim 7 in an imaging analysis in a patient.

20. The method as claimed in claim 19, wherein the imaging analysis is based on at least one of X-ray computed tomography (CT), magnetic resonance tomography (MRT), positron emission tomography (PET), single photon emission computed tomography (SPECT) and optical imaging such as near infrared fluorescence (NIRF).

21. The method as claimed in claim 11, wherein a positive and a negative selection are carried out in at least one of step (c) and step (d).

22. The method as claimed in claim 15, wherein the second phage surface protein is gpVIII.
